(19) United States
(12) Patent Application Publication
(10) Pub. No.: US 2024/0115412 A1
(43) Pub. Date: Apr. 11, 2024

(54) Male protector intended to contain emissions and/or leaks of fluids originating from the penis

(71) Applicant: Emiliano Andrés Meroi, Buenos Aires (AR)

(72) Inventor: Emiliano Andrés Meroi, Buenos Aires (AR)

(21) Appl. No.: 18/481,503

(22) Filed: Oct. 5, 2023

(30) Foreign Application Priority Data
Oct. 5, 2022 (AR) P20220102686

(51) Int. Cl. A61F 5/453; A61F 5/44

(52) U.S. Cl. CPC A61F 5/453; A61F 5/4401; A61F 2005/4402

(57) ABSTRACT

Male protector for containing emissions and/or leaks of fluids originating from the penis, comprising: —a first cylindrical part, shaped like a sheath, with a longitudinal lateral cut in axial direction and a fastening band at its proximal end, arranged on the circumference of the cylinder, composed of a supporting elastic and a closure means; and—an apical capsule, attached to the first cylindrical part, closed at its other distal end.

### CROSS-REFERENCE TO RELATED APPLICATION

The present application claims priority to Argentine patent application P20220102686, filed Oct. 5, 2022, which is hereby incorporated by reference in its entirety.

### TECHNICAL FIELD

The present invention is applied in the field of male personal hygiene, specifically aimed at preventing and/or containing urinary or any other type of fluid emissions originating from the penis.

### BACKGROUND

Currently, the vast majority of protectors for male use intended to contain fluid emissions originating from the penis are basically made up of diapers or dressings similar to those for female use for menstrual flow, but changing the point of placement thereof. These protectors have the disadvantage, in the first case, diapers, of being uncomfortable, bulky and more associated with incontinence, and in the second case, dressings, of not completely guaranteeing the function thereof due to problems in their positioning, the latter being very impractical if the male underwear is a boxer type brief. As an example, document US2020206017A1 can be cited.

There are also a series of slightly more specific protectors that are placed apically on the penis, but they cannot guarantee an adequate and safe positioning to avoid or mitigate spills, in addition to having an uncomfortable attachment to the testicles by means of auxiliary straps. At the same time, they are impermeable in their outer layer, preventing air circulation.

Among these protectors, documents EP2653141A1 and JP2018196556A can be cited.

The male protector is placed over the penis, is anatomical in shape and presents an innovative form that allows overcoming the above mentioned disadvantages, guaranteeing the correct, safe, easy and permanent placement of the device for the duration of its use.

### BRIEF DESCRIPTION OF THE DRAWING FIGURES

FIG. 1 refers to a perspective view of the male protector.

FIG. 2 refers to a view of the male protector inside a transparent wrapper for presentation to the user.

FIG. 3 refers to a perspective view of the top of the male protector.

FIG. 4 refers to a cutaway view at the height of the apical capsule (6) of a preferred embodiment of the male protector with four layers.

FIG. 5 refers to a cutaway view along the longitudinal axis of the male protector.

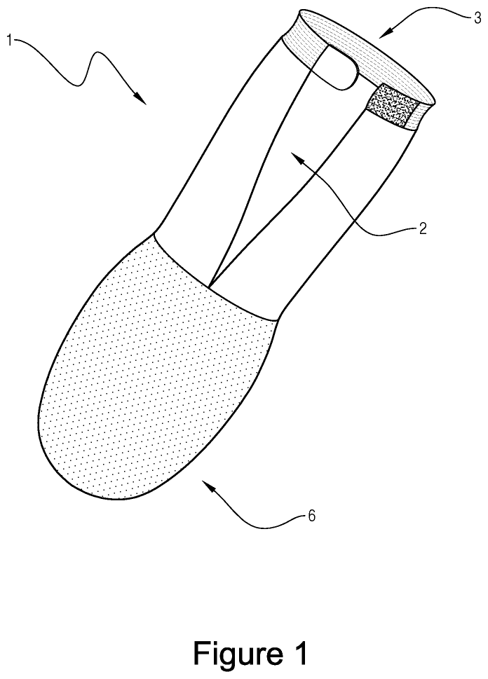

### DETAILED DESCRIPTION

The present invention relates to a male protector intended to contain emissions and/or leaks of fluids originating from the penis, on which it is placed.

Among the functions performed by the male protector are those of neutralizing odors, avoiding urine stains or post sexual intercourse flow emissions on both undergarments and outer garments, as well as containing drips and has the condition that it can be reused.

The male protector has two main parts:
a first part (1) and
a second part that we will call the apical capsule (6).

The first part (1), which is closer to the body of the user, has a predominantly cylindrical shape in the form of a sheath, through which the penis is inserted, with a lateral cut in axial direction and with a length that can range from one third to the entire length of the first part (1). Said cut has the purpose of being able to adjust the male protector to the different anatomies of the users and to make fitting easy for the user, without running the risk of wetting the first part (1). Said first part (1) may be constructed of a porous material composed of wood fiber, polyester fiber, bamboo fiber, or organic cotton, among others. These materials are not limiting in any way for the present invention and any suitable porous material that allows the free circulation of air may be used.

At the proximal end of the cylindrical part described above, a fastening band (3) is annularly located to keep the male protector fastened to the penis by means of a pressure adjustment. The fastening band (3) is constituted by a low resistance elastic support (4) that covers the entire perimeter of the cylindrical part and a fastening means (5) that has the purpose of keeping the male protector in position.

The closure means (5) may be a buckle closure or a reusable adhesive strip among others. These examples of closure means are not limiting in any way for the present invention and any other suitable closure means may be used, considering materials that do not irritate the skin of the user.

The second part of the male protector, which we will call the apical capsule (6), is attached to the first part (1), and is located from the shaft of the penis to its tip. Its purpose is to absorb liquids.

The junction between the first part (1) and the apical capsule (6) can be made by sealing, gluing or sewing.

The apical capsule (6) may have a predominantly cylindrical, spherical or drop-shape, with its distal end closed in such a way as to completely cover the surface of the glans.

The apical capsule (6) consists of at least three layers:
the first layer (7), which is in direct contact with the glans, is a soft, stay-dry textile cover made of polypropylene/polyester and polyethylene/viscose keeping the wearer dry and comfortable;
the second layer is the absorbent core (9), composed of paper pulp, a combination of paper pulp and/or super absorbent polymers that absorbs and retains fluids; and
the third or back layer (10), which is in contact with clothing. It is made of a polyethylene sheet that allows air circulation, but not the passage of liquids.

The materials listed above that constitute the various layers that make up the apical capsule (6) are not limiting, and can be any other material that meets the requirements expressed.

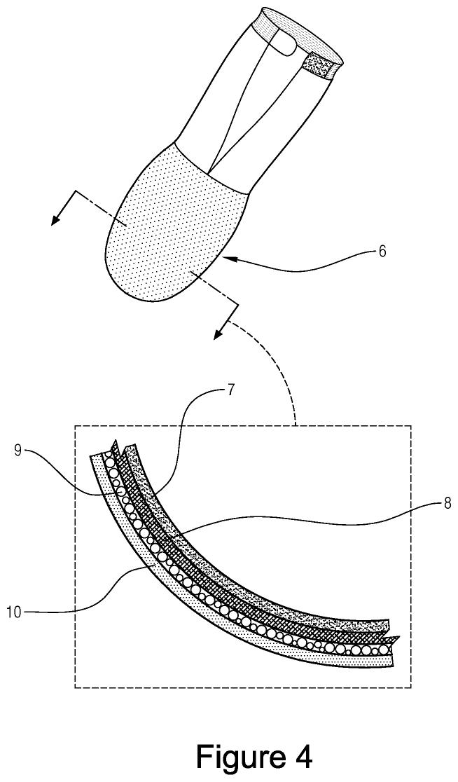

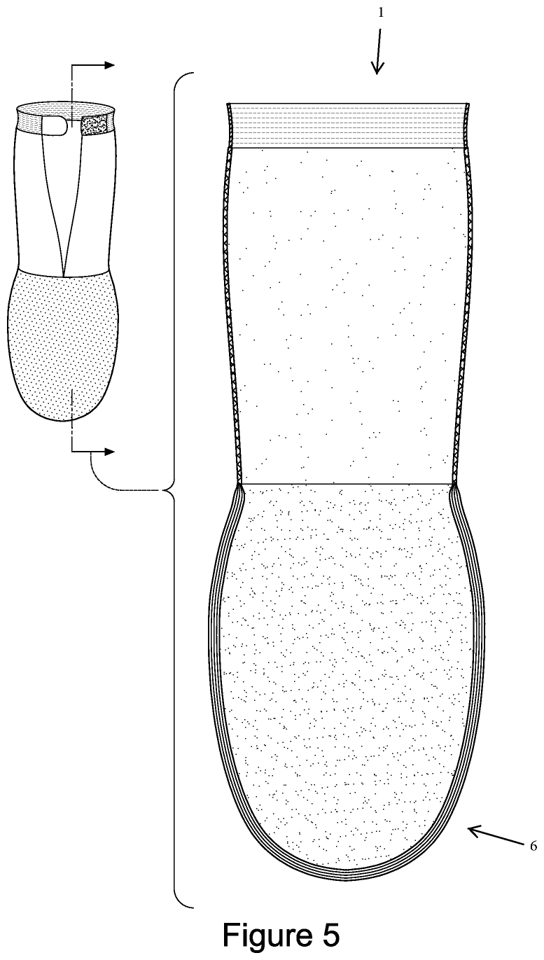

In a preferred embodiment (FIG. 4), there may be an additional layer (8) called acquisition layer, made of a porous material composed of wood fiber or polyester fiber, which conducts the collected fluid to the absorbent core.

These layers are bonded together by the function of the adhesive composed of various polymers or synthetic resin.

Given the high absorption capacity of the male protector, it can be “reused” several times until the absorbent material is saturated. In addition, the closure means (5) allows a quick and efficient repositioning of the male protector.

By way of illustration only, the overall length of the male protector may be in the range of 8 cm to 12 cm and the inner diameter of the protector may be in the range of 5 cm to 9 cm. These ranges are merely exemplary and in no way limit the present invention.

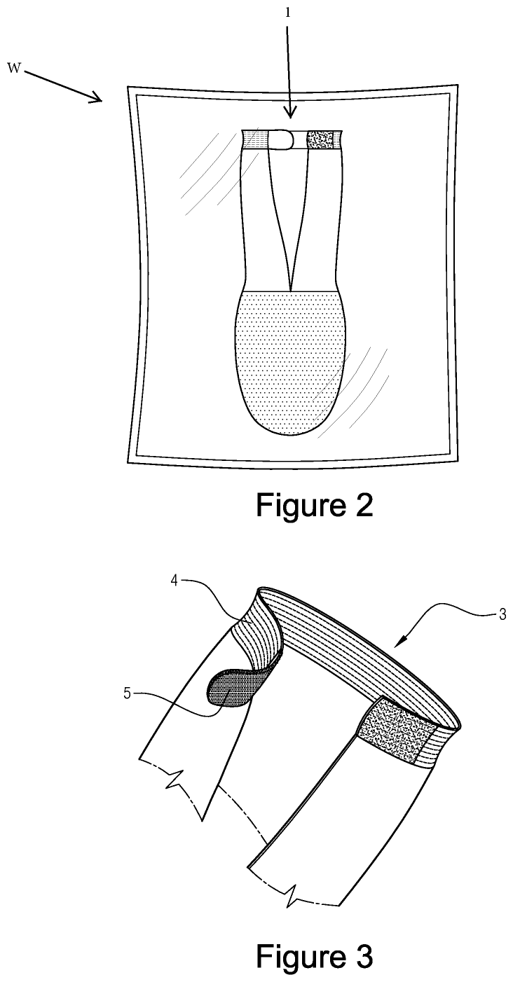

FIG. 2 refers to a view of the male protector inside a transparent wrapper (W) for presentation to the user.

1. Male protector to contain emissions and/or leaks of fluids originating from the penis characterized by comprising
a first cylindrical part, in the shape of a sheath, with a longitudinal lateral cut in axial direction and a fastening band at its proximal end, arranged on the circumference of the cylinder, composed of an elastic support and a fastening means; and
an apical capsule, attached to the first cylindrical part, closed at its other distal end.

2. The protector of claim 1, characterized in that the first part is formed of a porous material composed of wood fiber or polyester fiber.

3. The protector of claim 1, characterized in that the fastening means is a buckle fastener or an adhesive strip.

4. The protector of claim 1, characterized in that the apical capsule may be in the form of a cylindrical, spherical or drop-shaped sheath and consist of liquid-permeable, air-permeable and absorbent layers.

5. The protector of claim 4, characterized in that the liquid permeable, air permeable and absorbent layers are at least three layers: the first one is a textile layer, the second one is an absorbent layer of paper pulp or absorbent polymer and the third one is an air permeable but not liquid permeable layer, which are bonded together by an internal adhesive that may comprise various polymers or synthetic resins.

6. The protector of claim 1, characterized in that the bonding between the first part and the apical capsule is by sealing, gluing or stitching.
